(19) United States
(12) Patent Application Publication
(10) Pub. No.: US 2008/0171989 A1
(43) Pub. Date: Jul. 17, 2008

(54) Trans Urinary Bladder Access Device and Method

(76) Inventors: Stephen G. Bell, Rome (IT); Wayne A. Noda, Mission Viejo, CA (US); Elbert Y. Tzeng, Irvine, CA (US)

Correspondence Address: SCOTT EVANS, 1252 COUNTRY HILLS DR., SANTA ANA, CA, 92705, US

(21) Appl. No.: 11/869,021

(22) Filed: Oct. 9, 2007

Related U.S. Application Data
(60) Provisional application No. 60/885,133, filed on Jan. 16, 2007.

(51) Int. Cl. A61M 25/06; A61M 25/04

(52) U.S. Cl. 604/170.02; 604/164.01; 604/167.06; 604/170.01; 604/164.04

(57) ABSTRACT

The present invention relates to a device and method for accessing an anatomical cavity using a natural body orifice where penetration of a tissue wall is required. In particular, an objective of this invention is to provide a system for delivering an access device to the abdominal cavity through the bladder. An access device is described suitable for introduction into the bladder, at least partially disinfecting the access site, accessing the abdominal cavity through a wall of the bladder and insufflating the bladder and the abdominal cavity as required. The access device may be used to deliver an endoscope or other diagnostic or therapeutic instruments into the abdominal cavity.

### FIELD OF THE INVENTION

The invention relates to a device and related method for accessing an anatomical cavity in the body through a natural body orifice. More specifically devices and methods are described to access an anatomical cavity, more specifically the abdominal cavity, through the urinary bladder including sterilizing the bladder wall access site, inserting an access device through the wall and delivering instruments or endoscopes to the abdominal cavity.

### BACKGROUND OF THE INVENTION

Laparoscopic and endoscopic surgical procedures are minimally invasive procedures in which operations are carried out within the body by means of elongated instruments inserted through small entrance openings in the body. These procedures are less invasive leading to reduced surgical trauma, lower costs and quicker recovery times than conventional surgery. The initial opening in the body tissue, to allow passage of endoscopic instruments to an anatomical cavity in the interior of the body, may be a natural passageway of the body such as the vagina, urethra, esophagus or rectum or the opening may be created by a tissue piercing instrument such as a trocar, a scalpel or a needle through the skin.

However conventional laparoscopy through the skin of a patient does require extra time, anesthesia and potentially longer recovery times than natural body orifices. Many laparoscopic procedures performed today may be performed adequately and with improved results through these natural body orifices. However the tools, instruments and particularly the access devices required to access a natural orifices (NO) and penetrate into an anatomical cavity such as the peritoneum have been lacking. Particularly the ability to access an anatomical cavity from a remote natural body orifice that may be non-sterile or one that may be made non-sterile by the introduction of instruments from outside the natural orifice is often difficult. Furthermore, creating an instrument passageway that can accommodate various or multiple instruments through a small access site that also must be expected to provide insufflation for the abdominal cavity has not been available.

In the case of trans-cystic access through the urinary bladder wall, even though the bladder is considered a sterile environment, the sterility of the bladder may be compromised by the use of instruments that are introduced into the urethra and must travel to the bladder wall for introduction into the abdominal cavity. Furthermore the introduction of instruments through the bladder wall must insure that contamination is not carried into the peritoneum by these instruments.

Additionally, the access through the bladder wall may require the use of novel tissue penetration techniques that may separate tissue planes rather than cut tissue. Procedures employing such techniques may permit access to anatomical cavities and then be withdrawn without requiring closure devices such as staples or stitches. These closure devices greatly increase the time and complexity of NO surgery and their elimination may simplify the procedure, reduce surgery times and promote the use of NO related surgery.

Additionally the blind introduction of potentially sharp instruments into anatomical cavities through the cavity walls could potentially damage other tissues or organs that are located on the opposite side of the anatomical wall. These tissues or organs may require special access techniques and devices that control the depth of penetration of the access device into the anatomical cavity. These access devices may increase the safety of the initial cavity access so that other instruments or tools may be safely introduced into the body cavities as described. Additionally because the access site puncture should be as small as possible to avoid secondary closure devices, the access device may require that several working lumens or functions be integrated into a single device to efficiently use space and avoid time consuming instrument exchanges through the bladder.

Also to reduce the diameter of the incision in the wall the device will have a small incision footprint followed by dilation to accept the full diameter of the device.

A need exists therefore for improved anatomical access and in particular NO access devices that meet the needs described above. In particular, a need exists for such devices and techniques that can safely traverse a first layer of tissue, such as the wall of the bladder, gastro-intestinal tract, vagina or uterus or other organ or structure and penetrate into an anatomical cavity such as the abdominal space, an organ such as the gall bladder or pancreas, a blood vessel, spinal/vertebral/joint or into another hollow organ, hollow body, luminal structure, duct, vessel, or soft tissue structure.

### SUMMARY OF THE INVENTION

The present invention may solve the needs in the art stated above and may provide certain advantages over the prior art. The present invention may improve upon other available NO access devices and permit performing multiple techniques with multiple instruments through abdominal cavity access points, particularly the urinary bladder.

One embodiment of the present invention may be an access device for accessing an anatomical cavity of the body by penetrating a wall of an adjacent organ or tissue. The device may be an access device whereby the penetration of the wall may be performed using this device and using a method that does not compromise the natural sterility of the cavity. This device may have distal and proximal end portions including an elongated member with a lumen extending longitudinally inside. The device may have a first expandable member positioned at the distal end portion, said first expandable member adapted to expand from a non-expanded condition to an expanded condition whereby the cross sectional area is larger than the elongated member. The distance between the first expandable member in the expanded condition and the distal end portion of the device may control the depth of penetration of the elongated member through the wall of the anatomical cavity.

In one embodiment of the invention, the device may include a hollow obturator slidably disposed within the center lumen of the elongated member and the obturator may have a penetrating member at the distal end. Furthermore the elongated member may have a sealing member that may be adapted to provide a fluid tight seal between the outside diameter of the obturator and the inner diameter of the elongated member.

In another embodiment of the invention, the penetrating member may comprise an atraumatic tip that is configured to separate anatomical tissue or muscle layers so as to penetrate the anatomical cavity wall by spreading the tissue or muscle layers without cutting the muscle or tissue.

In another embodiment of the invention, the access device may be designed so that the distance between the first expandable member in the expanded condition and the distal end portion of the elongated member may be adjustable depending on the amount of expansion of the expanding member. Furthermore the expansion member may be a balloon where the size of the balloon may be controllable by the amount of inflation of the balloon. Alternately the expansion member may be a mechanical spring, mesh, web, plate or other mechanical device that can unfold, spring out, or otherwise expand in diameter.

In another embodiment of the invention, the access device may include a second expandable member. The second expandable member may be positioned distal of the first expandable member on the outside of the elongated member and be designed to expand inside the cavity and seal around the anatomical wall and the elongated member. This second expandable member may be alternately coupled to a distal end portion of an inner sheath that may be slidably disposed within the lumen of the elongated member. This sheath may advance into the anatomical cavity so that the expanded expandable member seals around a bladder wall and the elongated member.

In a further embodiment of the invention, the access device may include a removable outer sheath that may be disposed over the length of the elongated member. This sheath may have a closed distal end that is adapted to be penetrated by pushing the elongated member or an obturator distally relative to the sheath. Furthermore this sheath may have a closed distal end that is at least partially perforated and thereby adapted to open up when the elongated member or obturator is moved distally through it.

Another embodiment of the invention may include a method of accessing an anatomical cavity through a wall of an adjacent organ or tissue. The method may include positioning the tip of a hollow elongated member, which has a hollow obturator positioned inside, against a portion of the outer region of the organ wall. The obturator may have a sealing member adapted to provide a fluid seal between the obturator and the inner diameter of the elongated member. The method may further include expanding a first expanding member positioned at a distal end portion of the elongated member and sterilizing at least a portion of the wall by injecting a sterilizing fluid through the hollow portion of the obturator to fill an open space inside the hollow elongated member. The method may further include advancing the obturator within the elongated member to reduce the open space and thereby force the sterilizing fluid through the tip of the elongated member and into and around a portion of the outer region of the anatomical wall.

The method may further include penetrating the wall with the obturator and advancing the elongated member into the anatomical cavity until the expanded first expanding member abuts the outer region of the anatomical wall. This method may further include utilizing the distance between the expanded first expanding member and the distal end portion of the elongated member to control the penetration depth of the elongated member into the anatomical cavity. Another embodiment of the invention may include a method of expanding a second expanding member positioned distal of the first expanding member and near the distal end portion of the elongated member such that when the first expanding member abuts the outer region of the anatomical wall, the second expanding member abuts the inner region of the anatomical wall. The method may include sealing around the wall and the elongated member to inhibit fluid leakage at the penetration site and/or inhibiting distal and proximal movement of the elongated member.

A further embodiment of the invention may include a method of penetrating an anatomical wall by advancing an obturator through tissue planes without cutting the tissue. This method may further include sequentially advancing the obturator until the obturator tip separates at least one tissue plane aligned in one axis, rotating the obturator tip until the obturator tip separates at least one tissue plane aligned in a different axis and repeating these steps until access to the anatomical cavity is complete.

A further embodiment of the invention may include a method of performing trans-cystic urinary access of an abdominal cavity by positioning a tip of a hollow elongated member against a wall of the bladder, disinfecting at least a portion of the bladder by infusing a disinfecting fluid through the hollow portion of the elongated member, insufflating the bladder with gas and puncturing the wall with a piercing member and then advancing the elongated member into the cavity. This method may further include delivering the piercing member to the bladder wall through a working lumen of an endoscope or through a utility lumen of the elongated member. The method may further include directly visualizing the steps of puncturing the wall and advancing the elongated member with an endoscope. Furthermore the abdominal cavity may be insufflated by infusing a gas through the hollow portion of the elongated member.

It is understood that both the foregoing general description and the following detailed description are exemplary and explanatory only and are not restrictive of the invention as claimed. The accompanying drawings illustrating an embodiment of the invention and together with the description serve to explain the principles of the invention.

### BRIEF DESCRIPTION OF THE DRAWINGS

FIG. 1 is a sectional view of an access device of the present invention.

FIG. 2 is a detail drawing of an access device of FIG. 1 in proximity to an anatomical wall.

FIG. 3A is a perspective view of an embodiment of the present invention showing the infusion of a disinfecting fluid.

FIG. 3B is a detail view of FIG. 3A showing disinfecting fluid near an anatomical wall.

FIG. 3C is a detail view FIG. 3B showing the forward advancement of an obturator.

FIG. 4A is a perspective view drawing of an embodiment of the present invention showing an expanding member.

FIG. 4B is a perspective view drawing showing the device of FIG. 4A through the anatomical cavity wall.

FIG. 5 is another perspective view drawing showing the device of FIG. 4B with a second expandable member deployed.

FIG. 6 is a sectional view of an alternative embodiment of an expandable member.

FIG. 7A is perspective view of an alternative embodiment showing an access sleeve.

FIG. 7B is an end view of the device of FIG. 7A.

FIG. 7C is perspective view of the device of FIG. 7A with the end portion perforated.

FIG. 8A is a perspective view of an embodiment of the present invention including an end cap.

FIG. 8B is an end view the end cap of FIG. 8A.

FIG. 8C is a perspective view of the device of FIG. 8A disposed at the anatomical wall and having a penetrating member.

FIG. 8D is a perspective view of the device of FIG. 8A shown positioned inside the anatomical cavity.

FIG. 9A is a cutaway view of a sterile sheath embodiment of the present invention.

FIG. 9B is a cutaway view of the embodiment FIG. 9A showing the sheath accessing the anatomical cavity and the introduction of an instrument.

FIG. 9C is a cutaway view showing the sheath expanded into the anatomical cavity.

FIG. 10A is a perspective view of one embodiment of a penetrating member.

FIG. 10B is a side view of the penetrating member of FIG. 10A.

FIG. 10C is a perspective view of another embodiment of a penetrating member.

FIG. 11A is a drawing showing planes of tissue arranged in multiple axis.

FIG. 11B is a drawing showing a penetrating member aligned with a first plane of tissue.

FIG. 12A is a drawing of an embodiment of the current invention, an access device, positioned in a bladder with one expanding member expanded.

FIG. 12B is a drawing of the access device of FIG. 12A with a second expanding member expanded and the bladder distended.

FIG. 12C is a drawing of the access device of FIG. 12B with a penetrating device positioned in an anatomical cavity.

FIG. 13A is a drawing of an alternate embodiment of the current invention showing two channels positioned at the end portion of an access device.

FIG. 13B is a drawing of the embodiment of FIG. 13A showing the two channels extended form the access device and separated.

FIG. 14A is a drawing of an alternate embodiment of the current invention showing an introducer sheath.

FIG. 14B is a drawing of the sheath of FIG. 14A with the balloon inflated inside the bladder.

FIG. 14C is a drawing of an alternate embodiment of the elongated member

FIG. 14D is a drawing of the elongated member of FIG. 14C positioned inside the introducer sheath.

FIG. 14E is a drawing of the system of FIG. 14D with a piercing member positioned in the utility lumen.

FIG. 14F is a drawing of the piercing member across the wall of the bladder.

FIG. 15A is a drawing of another alternate embodiment of the elongated member.

FIG. 15B is a drawing of the elongated member of FIG. 15A shown inside a bladder.

FIG. 15C is a drawing of the elongated member of FIG. 15A with the piercing member extended through the bladder wall.

FIG. 15D is a drawing of the elongated member of FIG. 15A showing insufflation of the bladder and abdominal cavity.

FIG. 15E is a drawing of the elongated member in the abdominal cavity with the piercing member removed.

FIG. 16 is a drawing of an alternate embodiment of the distal end portion of the elongated member.

### DETAILED DESCRIPTION OF THE PREFERRED EMBODIMENTS

Although several different methods of accessing anatomical cavities in the body have been described and used, particularly laparoscopic procedures that access the abdominal cavity through the skin, a better least invasive device and method for performing least invasive procedures through natural body orifices is needed. These procedures take advantage of the natural orifice so that no outward incision or scar is necessary. These procedures also may require less recovery time and may be quicker to perform.

However accessing body cavities through the esophagus, rectum, uterus, bladder or other natural body orifices (NO) has other challenges. Devices and methods that utilize these points of entry into the body are often entering a sterile environment such as the peritoneum from a non-sterile or potentially non-sterile environment. Therefore it is important that the initial access site be sterile and that the site remains sterile after the procedure has been completed and the device has been removed. This is particularly important when the initial access device creates a conduit through which other instruments or interventional devices may be inserted.

Secondly it is important to access these natural body orifices with access devices and accompanying methods that have small diameters. Some NO are small and the pathway to reach them may be relatively small compared to general surgery or laparoscopic interventions. This necessitates small diameter instruments and access devices. Additionally the smaller the puncture site into the anatomical cavity, the smaller the chance of fluid leakage and associated risk of infection.

Furthermore, it may be important to create a stable platform that can be used by other instruments to perform a procedure. This may require that the access device be capable of coupling or attaching at least temporarily to at least a wall of the cavity. This may prevent the access device from pulling out from the wall prematurely or from being accidentally advanced into the cavity itself. Also a device that efficiently utilizes space or reduces the number of instrument exchanges required may save the operator time.

Finally many cavities of the body are positioned close to other critical body structures, cavities, lumens or organs. It is important that these nearby structures are not inadvertently punctures or damaged. This is often difficult because some NO access techniques are performed “blind”. That is to say the operator may not be able to directly or even indirectly (via endoscopy, fluoroscopy or X-ray) see the surrounding structures. Therefore it may be important to have a device that provides adequate insufflation capabilities and controls the penetration through the anatomical cavity wall. This may include features that control or limit the direction of the access or the depth that the device protrudes through the wall of the cavity. Additionally the configuration of the portion of the device that enters the cavity may be important.

It is believed that the various embodiments of the current invention may solve these challenges as described.

In this application an emphasis on trans-cystic or access to the abdominal cavity through the urinary bladder is typically described, however this is not meant to be limiting because the designs, features and benefits of this device and method are meant to apply to any point in the body where access to an anatomical cavity is desired. This may include anatomical cavities such as the abdominal cavity, pleural spaces, gastrointestinal tract, reproductive tract, the kidney or bladder or other vessels organs, ducts or spaces in the body. Likewise the features of this invention are not limited to just access devices but should be considered for any device that utilizes a method to access these cavities or spaces including primary or secondary interventional devices that may not require a separate access device but that may be inserted directly into these spaces or cavities.

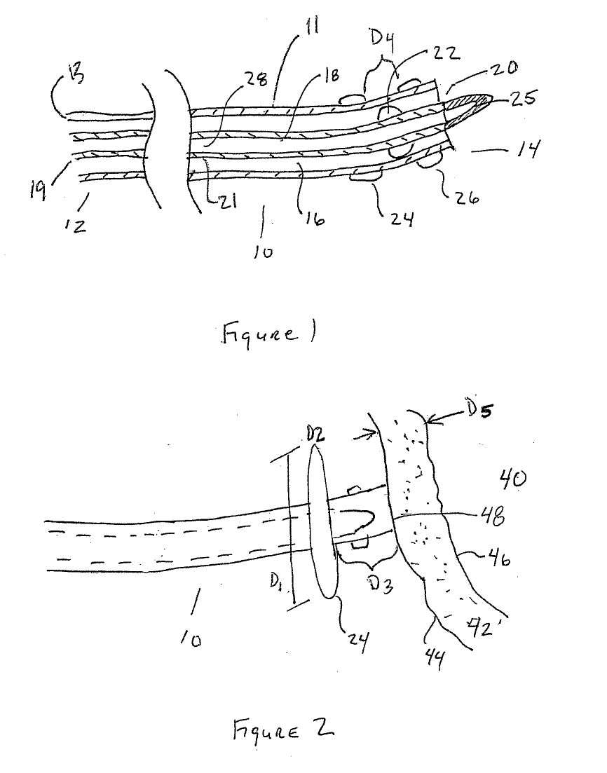

An embodiment for accessing the anatomical cavity is described in FIG. 1. An access device 10 having a proximal end 12 and a distal end 14 is shown. It is comprised of a hollow elongated member 11 having an inside lumen 16 extending longitudinally from the proximal end 12 to the distal end 14. The elongated member may be a cannula, sheath, tube, trocar or catheter or any other tubular structure suited for placement through a NO. The elongated member 11 may be flexible or rigid and is shown with a single wall thickness 13. This wall may be a polymer, metal or shaped memory alloy and is constructed using techniques and materials known to one skilled in the art. The elongated member may be steerable by the operator by actuating an actuator (not shown) at the proximal end 12 of the elongated member 11.

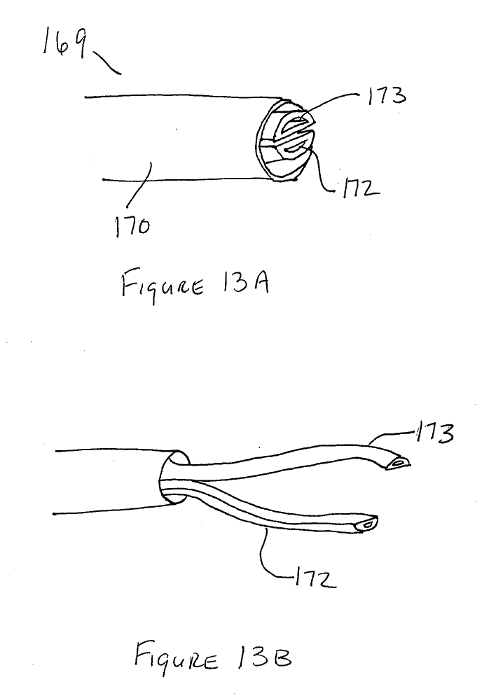

The elongated member 11 may have a secondary elongated member 18 disposed within the inside lumen 16. The secondary elongated member 18 may be a cannula, obturator, needle or stylet. By way of example this secondary member 18 will be referred to as an obturator. The obturator 18 has a proximal end portion 19 and a distal end portion 20, an outside wall surface 21 and may include an inside lumen 28. The obturator is adapted to be positioned inside the inside lumen 16 of the elongated member 11 so that the obturator 18 can be moved in a proximal or distal direction within the inside lumen 16. The obturator 18 may include a sealing member 22 formed or coupled to the outside wall 21 of the obturator 18 as shown at the distal end portion 20 or may be positioned elsewhere along the obturator wall 21. Alternately this sealing member 22 may be formed or coupled to the inside wall of the elongated member 11. This sealing member is adapted to form a fluid tight seal between the inside lumen 16 of the elongated member and the outside wall 21 of the obturator 18. This seal 22 is particularly designed to prevent fluid, even fluid under pressure, from leaking from a space S located distal to the end of the obturator 20 between the obturator and the inside lumen. This seal may be inflatable or expandable from a first non-expanded condition such as an inflatable bladder or balloon or may be a static seal such as an O-ring, membrane, pressure fit or septum that provides a continuous sealing function. The distal end portion 20 may include a penetrating member 25 that is adapted to penetrate tissue planes while minimizing or eliminating the need for cutting tissue at the penetration site. This feature will be described in more detail in FIGS. 11A and 11B.

The elongated member 11 may include at least one expandable member positioned at the distal end portion 14 of the elongated member. As shown in FIG. 1, a first expandable member 24 is shown positioned at the distal end portion 14. This expandable member 24 may be formed as a part of or otherwise coupled to the outside wall of the elongated member 11. The expandable member may have a first condition where member is essentially un-expanded and the outside diameter of the expandable member 24 is substantially the same as the outside diameter of the elongated member 11. The expandable member 24 may have a second condition whereby the member 24 expands so that the member has a cross sectional area substantially larger than said elongated member. The member 24 may be self expandable and expand from the first condition to the second condition with the removal of a constraining sheath. Similarly the member 24 may be self expandable and expand from the first condition to the second condition by the activation of a shape memory alloy such as Nitinol. This material expands to a pre-determined size and shape when heated to a certain temperature or released from a constrained position to return to its natural position. The member 24 may also be expanded from the first condition to the second condition by inflating the member 24 with a fluid or by inflating a balloon positioned under the expandable member. The member 24 may also be expanded from the first condition to the second condition by activating a pull wire or manipulating the expandable member by shortening its length such as with a mesh or stent. Preferably the member 24 is a balloon and can be inflated by infusing a fluid into a connector (not shown) at the proximal end of the device 10. This connector is in fluid communication with an inflation lumen (not shown) that communicates with the interior of the expandable member such that the amount of infusate may be regulated by the operator. If the balloon was made from an elastic rather than inelastic material, then by regulating the amount of infusate, it is conceivable that the size of the expandable member 24 can be controlled. If an elastic balloon is calibrated prior to introduction into the NO, the operator can determine the approximate size of the balloon 24 even when the balloon is inside the NO and hidden from the operator. When the size of the balloon is referenced that is meant to include both the outside diameter D1 and/or the width of the balloon D2 as shown in FIG. 2. The balloon may be fabricated such that the outside diameter D1 and the width D2 of the balloon is variably controllable. An example of this may be a balloon made out of a partially elastic material such as a partially irradiated elastic balloon material. Alternately the balloon 24 may have a fixed dimension and be inelastic so that the balloon size is constant independent of the infusate amount. In a preferred embodiment, a balloon 24 may be adapted to have a thickness D2 that is variably controllable so that the effective distance D3 between the balloon 24 and the distal end 14 of the elongated member 11 can be controlled by the amount of fluid infused into the balloon 24.

The elongated member 11 may include a second expandable member 26 positioned at the distal end portion 14 of the elongated member and distal from the first expandable member 24. As shown in FIG. 1, a second expandable member 26 is shown positioned at the distal end portion 14. This expandable member 26 may be formed as a part of or otherwise coupled to the outside wall of the elongated member 11. The expandable member may have a first condition where member is essentially un-expanded and the outside diameter of the expandable member 26 is substantially the same as the outside diameter of the elongated member 11. The expandable member 26 may have a second condition whereby the member 26 expands so that the member 26 has a cross sectional area substantially larger than said elongated member. The other characteristics of the second expandable member 26 include the scope and variations described for the first expandable member 24. The distance D4 between the first and second members may be fixed or variable depending on the type of balloon employed and the amount of fluid infused into the first and second expandable members. Preferably the distance between the expandable members D4 is approximately equivalent to the thickness D5 of the anatomical cavity wall 42 shown in FIG. 2. As will be shown this distance D4 may be helpful to stabilize the device 10 and seal the access site from leakage.

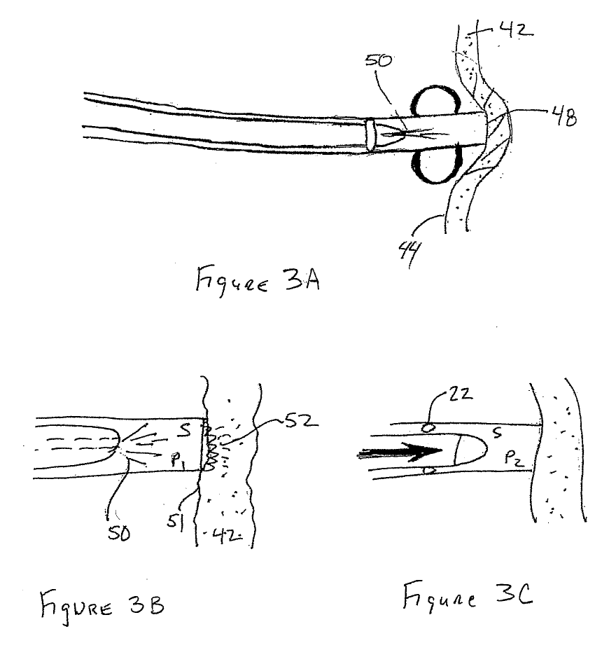

In one embodiment of the invention the device 10 may be used to provide an improved method to access an anatomical cavity. The access device 10 including the elongated member 11 and the obturator 18 is first positioned near an anatomical cavity 40 having an outer region 44 and an inner region 46 and a wall thickness 42. The distal end portion 14 is brought near the outer region 44 until the distal end 14 of the device 10 contacts the outer region 44. As shown in FIG. 3A, the device 10 may be advanced against the outer region 44 such that a deflection of the wall 42 is created by the distal end portion 14. This firm abutment of the device and the wall 40 may help to seal the device end at the wall 42. The first expanding member 24 is expanded as previously described. The obturator 18 is positioned near the distal end portion 14 of the elongated member 11 for the initial introduction into the NO and positioning at the outer region of the cavity. As shown in FIG. 3B, once the first expandable member 24 is expanded, the obturator is partially withdrawn to create a space S inside the distal end portion of the elongated member 11.

A sterilizing fluid 50 is introduced into the inner lumen 28 of the obturator through a valve at the proximal end of the obturator 18 (not shown). This sterilizing fluid 50 exits at the open end of the obturator 18 and fills the space S. Because the obturator has a valve at the proximal end of the obturator 18, when the valve is closed the sterilizing fluid 50 cannot escape through the inner lumen 28 of the obturator and the sealing member 22 prevents leakage along the inner lumen 16 of the elongated member 11. The sterilizing fluid 50 is confined to the inner lumen of the obturator and space S. The fluid 50 may also be initially injected with a pressure P1 such that the fluid in the space S may infuse into any folds 51 in the outer region 44 of the cavity wall 42. Additionally the sterilizing fluid 50 may also permeate into the tissue 52 of the wall 42 such that the outer region 44 of the wall inside the device open lumen may be sterilized and furthermore at least a portion of the wall 42 may be sterilized.

In an alternate embodiment, the sterilization of the wall 42 may be further enhanced as the obturator is now advanced distally as shown by the arrow in FIG. 3C. Because the seals 22 prevent the sterilizing fluid from escaping through the inner lumen 16, the proximal end of the hollow obturator is closed with a valve and the distal end portion of the catheter abuts the cavity wall 42, the pressure P1 of the fluid 50 in space S increases to a new pressure P2 that is greater than P1. This increase in pressure may cause greater permeation of the sterilizing fluid 50 into the cavity wall 42.

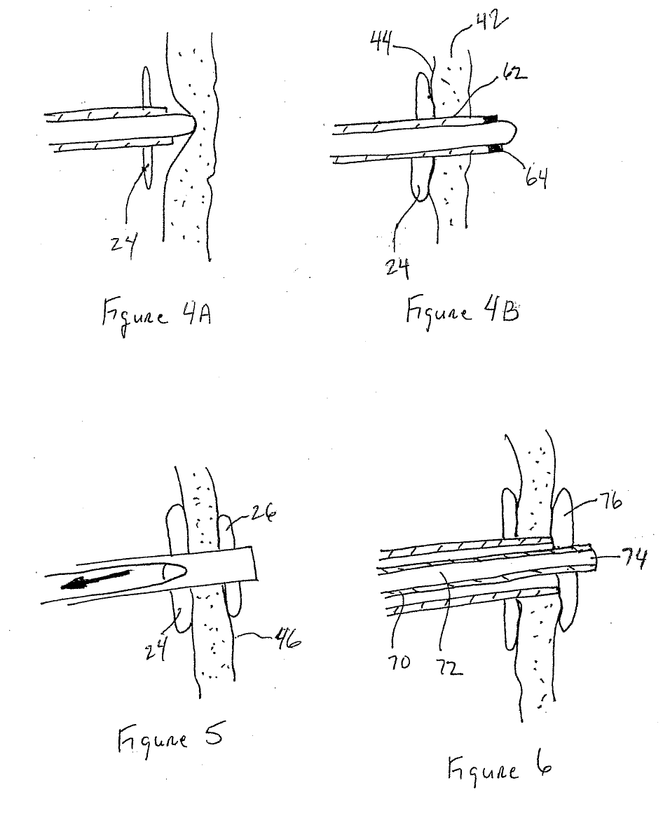

As shown in FIGS. 4A-B, the obturator is advanced until it is positioned such that the penetrating member 25 slightly protrudes from the distal end portion 14 of the elongated member 11. As the access device 10 is advanced distally, the penetrating member 25 penetrates the cavity wall 42. The depth of the device 10 penetration is controlled by the first expanding member 24. The device 10 penetrates the cavity 40 until the first expanding member 24 abuts with the outer region 44 of the cavity wall. The depth of the penetration may be further controllable by adjusting the width D2 of the expandable member 24. This width may be adjusted by controllably adjusting the amount of fluid infused into the expandable member. If the expandable member 24 is a balloon, then the width D2 can be adjusted by controllably adjusting the amount of fluid delivered to the balloon.

The penetration of the access device may be monitored by observing the distal end portion of the device using fluoroscopy. The distal end of the device may have a radiopaque marker 64 as shown in FIG. 4B so that the movement of the marker and the distal end portion 14 of the elongated member can be observed. In the case of an expandable member that is a balloon, the balloon can be inflated with a radiopaque fluid that can be visualized with fluoroscopy. Additionally the depth of penetration may be monitored by observing depth markings applied to the proximal end of the shaft elongated member.

As shown in FIG. 5, once the cavity has been penetrated, the second expandable member 26 may be expanded inside the cavity and alongside the inner region 46 of the cavity 40. In this configuration, the second expandable member 26 may seal around the elongated member 11 and the inner region of the cavity wall. This may inhibit the egress of cavity fluids out of the cavity 40 and alongside the outer diameter wall 62 of the elongated member 11. The first and second expandable members also facilitate securement of the access device at the access site. The first expandable member 24 may inhibit distal movement of the access device and the second expandable member 26 may inhibit proximal movement of the access device 11. Once securely positioned, the obturator 18 may be withdrawn and the open lumen of the elongated member used to introduce additional diagnostic or therapeutic instruments into the cavity.

An alternate embodiment of the invention is shown in FIG. 6. Once the obturator and the elongated member have penetrated the cavity wall 42, the obturator 18 may be withdrawn. A hollow inner sheath 70 having an inner lumen 72, a distal end 74 and a sheath expandable member 76 formed at or attached at the distal end may be introduced into the inner lumen 16 of the elongated member 11 and advanced until the distal end 74 of the inner sheath 70 exits the distal end portion 14 of the elongated member and enters the cavity 40. The sheath expandable member 76 may be expanded in a method similar to the first and second expandable members alongside the inner region 46 of the cavity 40. In this configuration, the sheath expandable member 76 may seal around the elongated member 11 and the inner region of the cavity wall. This may inhibit the egress of cavity fluids out of the cavity 40 and alongside the outer diameter wall 62 of the elongated member 11. The first expandable member 24 and the sheath expandable member 76 also facilitate securement of the access device at the access site. The first expandable member 24 may inhibit distal movement of the access device and the sheath expandable member 76 may inhibit proximal movement of the access device 11.

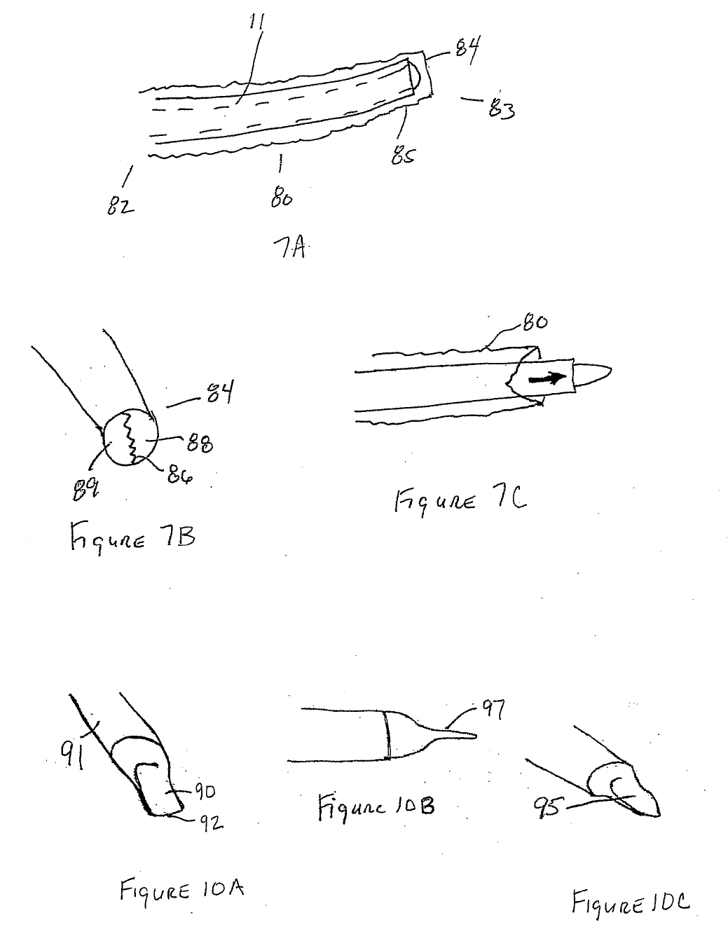

Another embodiment of the invention is shown in FIGS. 7A-C. An outer sheath 80 having an open proximal end 82 and a closed distal end 83 is disposed about the elongated member 11. The outer sheath 80 is designed to provide a sterile covering for the elongated member inside. As the member and sheath 80 are advanced to an access site such as the bladder through the urethra, the elongated member sterility is protected by the sheath 80. Once the elongated member and sheath are delivered to the intended access site, the elongated member can be extended distally relative to the sheath such that the distal end of the elongated member impinges on the closed end 84 of the sheath 80. The closed end 84 of the sheath 80 may have an opening feature 86. This feature may be a perforation or other weakened section of the closed end such that when the pressure is applied to the opening feature by the distal end portion of the elongated member, the opening feature splits into at least two sections 88 and 89 to accommodate the continued distal movement of the elongated member. This feature facilitates the sterile delivery of the elongated member to the access site and then permits the advancement through the sheath when required as shown in FIG. 7C.

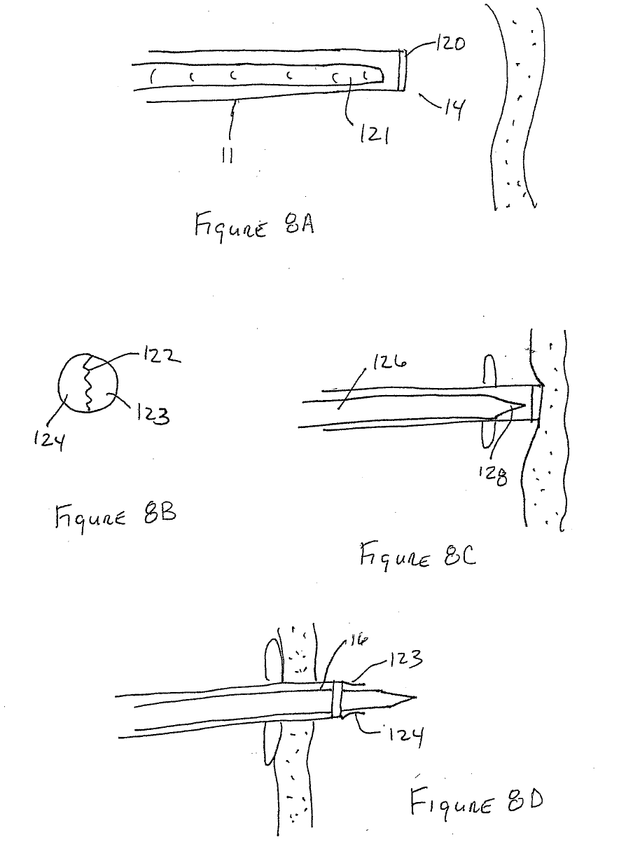

FIGS. 8A-D illustrate another embodiment of the invention where an elongated member 11 has a closed end 120 at the distal end portion 14. The closed end may function similarly to the sheath described previously. The closed end 120 may be clear so that an endoscope 121 may be brought adjacent the closed end visualize through the closed end to guide the elongated member 11 to the access point. The endoscope 121 or an obturator 126 may be advanced distally from the elongated member 11 to split open the closed end 120 if desired. The closed end may have an opening feature 122. This feature may be a perforation or other weakened section of the closed end such that when the pressure is applied to the opening feature 122 by the distal end portion of the endoscope 121 or an obturator (not shown), the opening feature splits into at least two sections 123 and 124 to accommodate the continued distal movement of the endoscope or obturator. Alternately, the device may be configured with an obturator 126 having a sharpened tip 128. In this configuration shown in FIG. 8C, the endoscope may be used to guide the elongated member 11 to the access point and then be exchanged for the obturator 126. With the closed end 120 abutting a wall of the bladder, distal advancement of the sharpened tip obturator will penetrate the closed end and the bladder wall. Once the obturator has pierced the wall, the elongated member may be advanced over the obturator and into the abdominal space. This embodiment may reduce the potential for infection by limiting the exposure of the elongated member inner lumen 16 to the inside of the bladder. Such exposure would be expected with a design having an open distal end, especially as the elongated member is introduced through the urethra.

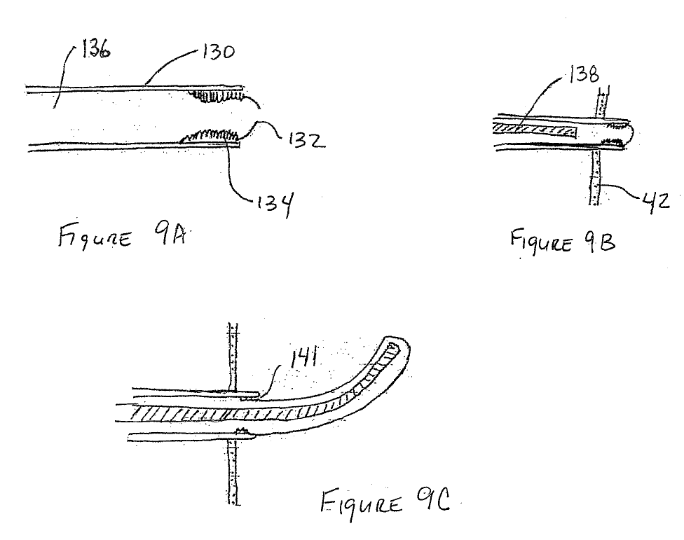

Another embodiment of the invention that is also designed to provide a means to introduce an instrument into an anatomical cavity such as the abdominal space in a sterile manner is shown in FIGS. 9A-C. In FIG. 9A an elongated member 130 is shown that has an inner lumen 136 and a collapsible sheath 134 coupled to the distal end portion of the elongated member inner lumen 136. The collapsible sheath 134 has a clear end portion 132 that is useful so that an endoscope 138 that abuts this clear end portion 132 may still view the anatomy outside the sheath. The collapsible sheath 134 is collapsed inside the inner lumen 136 but is designed to expand and unfold or uncurl once the elongated member is positioned inside the target body cavity such as the bladder. As shown in FIG. 9B, the elongated member is positioned through the bladder wall 42 and the scope 138 is advanced through the inner lumen 136 toward the distal end of the lumen. As the scope 138 is advanced further, the collapsible sheath 132 expands into the abdominal cavity but is still encapsulated by the sheath which maintains a sterile barrier for the inside of the elongated member and any instruments that may be passed through it. The sheath may be coupled to an inner wall of the inner lumen 136 at adhesion point 141. This coupling may be accomplished using adhesives, mechanical or thermal bonding or using other techniques well known in the art. The scope 138 is shown expanded inside the anatomical cavity in FIG. 9c. Alternately the sheath may have an opening feature as previously described so that an endoscope 138 or other instruments can be passed through the clear end portion 132 and extend beyond the sheath into the anatomical cavity of choice.

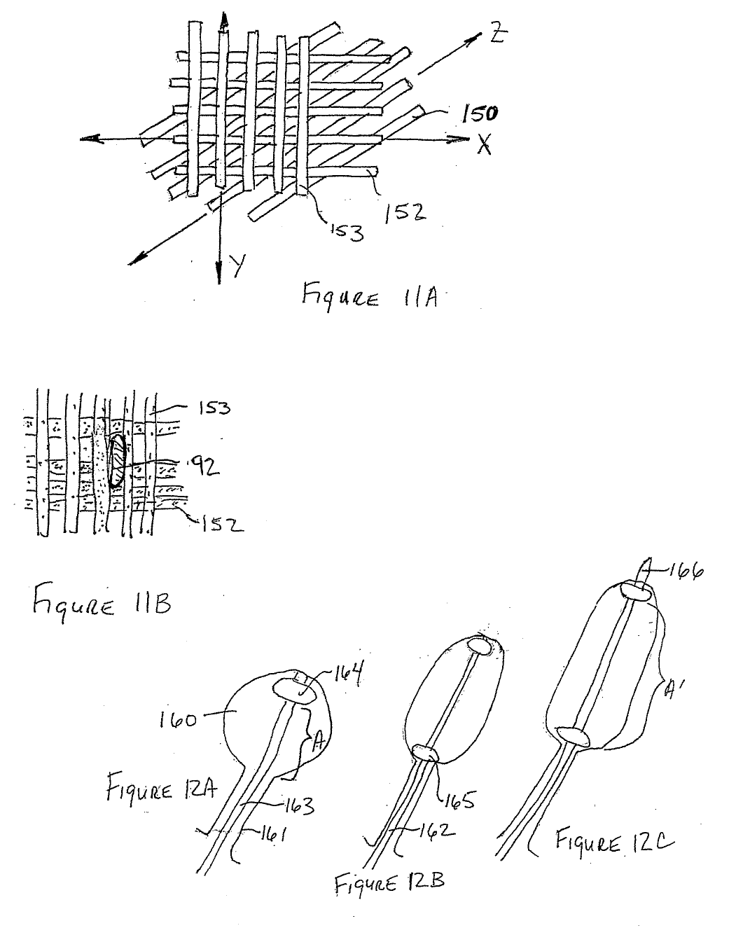

Referring now to FIGS. 10A-C, a blunt tip obturator 91 is shown that may be used for atraumatic entry through a tissue wall, preferably the bladder wall. The obturator may be advanced to the entry site using an elongated member as previously described. Once positioned at the access site, the blunt tip obturator 91 may be used to affect entry through a tissue wall without cutting through the tissue itself. The blunt tip is designed to spread apart tissue planes and slowly penetrate the tissue without artificially cutting tissue. The tip of the obturator 90 is fashioned like a knife but the distal end is flat without a sharp point. This blunt tip is designed to minimize the potential for cutting through tissue. The tip 90 is also wider in one direction than another. Side 97 is much thinner than the opposite side. This results in a flat, wide blunt tip tool that is suited for gently separating tissue planes without dissection. Alternately the obturator may have a tip that is also flat and wide but also having a slight pointed tip 95 as shown in FIG. 10C.

FIGS. 11A-B show a representative sample of the tissue found in the wall of the bladder. As shown for illustration only, the tissue is layered with three layers of tissue or tissue planes 150, 152 and 153. These planes are shown aligned in three axes, X, Y, and Z. The tissue found in the bladder wall may have more or less planes than shown here and the planes may be aligned in various orientations having different axis than that illustrated, It not the intent to accurately illustrate the tissue structure of all bladder walls but to illustrate that these walls may be constructed with a tissue structure that may be spread apart and traversed without the need to slice through this tissue. The advantage of this type of access may be that there is less trauma to the tissue wall, a faster healing time may result and most preferably that the tissue may better self-seal as the instruments or elongated member is withdrawn. This type of self-sealing should not require a secondary operation to staple, suture or otherwise close an access point. As the instrument is withdrawn the bladder walls would return to their original positions and ideally seal the access point so that abdominal fluids or gases don't enter the bladder and more importantly that potentially non-sterile fluids or gases don't egress from the bladder to the abdominal space. In FIG. 11B, a section view of the blunt flat tip of the obturator 92 is shown positioned between two tissue strands of tissue plane 153 aligned along axis Y. As this blunt tip is advanced deeper into the tissue, the tip 92, which is already past tissue plane 153, will abut against tissue plane 152 aligned along axis X. However if the tip 92 is rotated until the flat portion of the tip is aligned with the tissue strands of plane 152, the strands of plane 153 will be spread and the tip 92 will be positioned correctly to pass through plane 152. Further advancement of the tip deeper into the tissue will result in the tip encountering additional tissue planes such as plane 150 aligned along axis Z. The tip may be rotated and advanced repeatedly until the tip has successfully traversed the bladder wall and is inside the abdominal cavity. In this manner the tissue wall may be crossed atraumatically without the necessity for using a blade or scalpel to cut tissue planes.

Alternately the bladder wall may be crossed with the use of a dilating balloon sheath or trocar (not shown) using a penetrate and dilate approach. Using this method, the operator may insert a balloon slightly into the bladder wall and then dilate the balloon to spread apart tissue planes. As the balloon is dilated, the tissue spreads apart slightly allowing for a deflated balloon to penetrate deeper into the tissue wall. This process of penetration followed by balloon dilation followed by additional penetration and balloon dilation may be repeated until the balloon sheath is across the bladder wall. The balloon sheath may be withdrawn and exchanged for a dilator or the elongated member. Because tissue is not cut with this procedure, it is believed that the entrance site will seal upon withdrawal of the elongated member.

Access to the bladder may be difficult if the bladder has no tone and has become weak and distended which is common in the elderly population. In particular, accessing the bladder wall and placing instruments through a flaccid bladder wall may be difficult. In order to make the bladder wall tauter and firm it may be helpful to distend the bladder wall with a device illustrated in FIGS. 12A-C. An elongated member 163 is shown having a proximal end and a distal end and is positioned through the urethra 161 and inside the bladder 160. An inflatable balloon 164 is shown coupled to the distal end of the elongated member 163 and positioned against the distal wall of the bladder. The balloon 164 is inflated and shown separated from the proximal neck of the bladder by a distance A. An inflatable sheath 162 having an inflatable balloon 165 is next inserted into the bladder coaxially over the elongated member 163. The inflatable balloon 165 is inflated and positioned at the bladder neck such that it is wedged inside the bladder at the neck. The two balloons are moved away from each other by sliding the sheath 162 over the elongated member 163 in a proximal direction relative to the elongated member 163. As the balloons spread apart from each other to a new separation distance A′, the bladder lengthens and may be stretched slightly. This has the effect of elongating and tightening the bladder wall and provides a stable platform to insert an obturator 166 through the bladder wall and into the abdominal cavity.

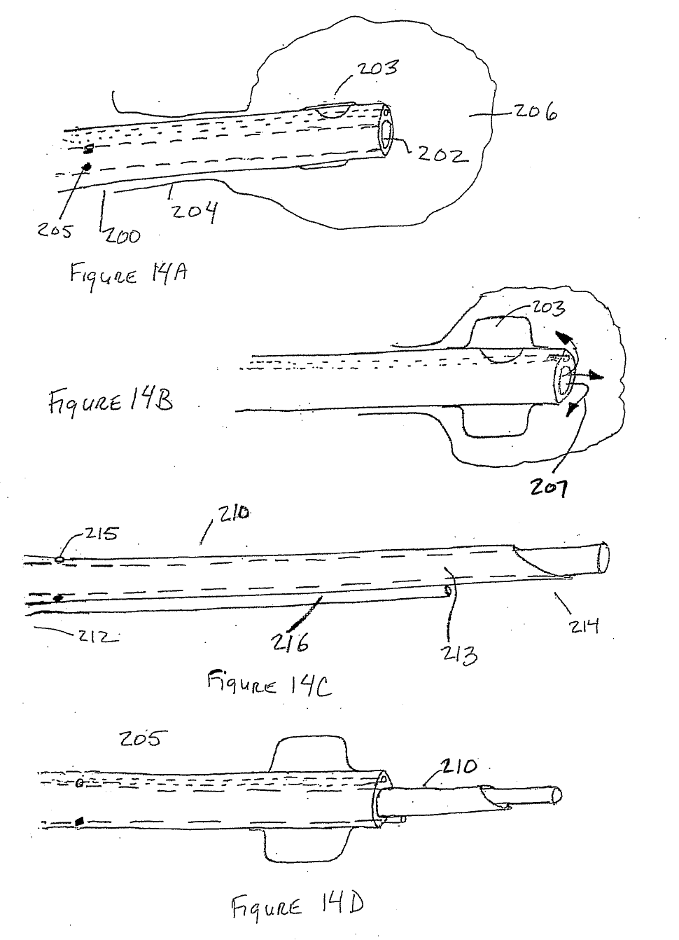

Another embodiment of the current invention is shown in FIGS. 13A-B. Maximizing the usage of space inside the elongated member or more particularly the working channel of an endoscope is particularly important especially when the size of the access site and the instruments that pass there through is very small. Additionally it is useful to have the ability to visualize and perform interventions using more than one axis. A single instrument can only operate in a limited area but if there are two instruments each having there own axis of operation then more can be accomplished. A device 169 is shown having a long elongate hollow member 170 with two smaller inner members 172 and 173 inside. These inner members 172 and 173 are configured to maximize the usage of the inner space of the elongated member and may have inner lumens inside. The inner members shown have a “D” shaped configuration which is often used to maximize usage of inner diameter space in a round inner lumen device. As these inner members are extended past the end of the elongated member, they can separate or split apart from each other so that their inner lumens may be used for the introduction of small instruments, scopes or tools into an anatomical space. These inner members may be independently steerable so that they may be guided to separate locations inside the anatomical cavity. With this independent capability, these inner members may provide the operator greater control over instrument placement and usage.

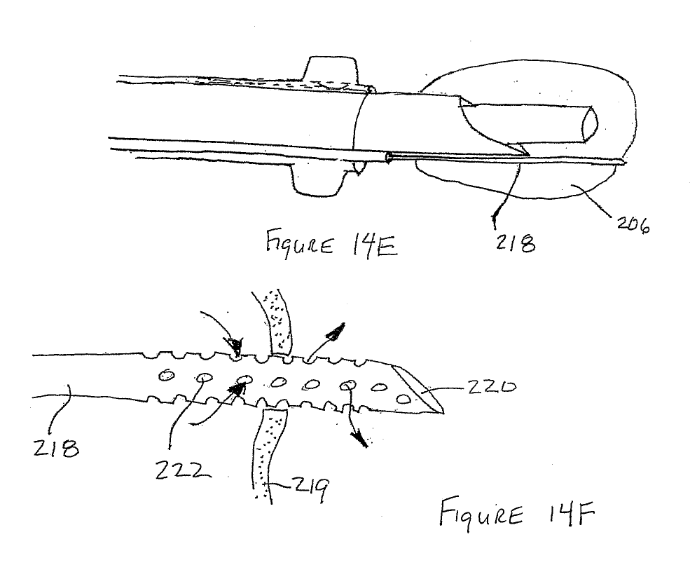

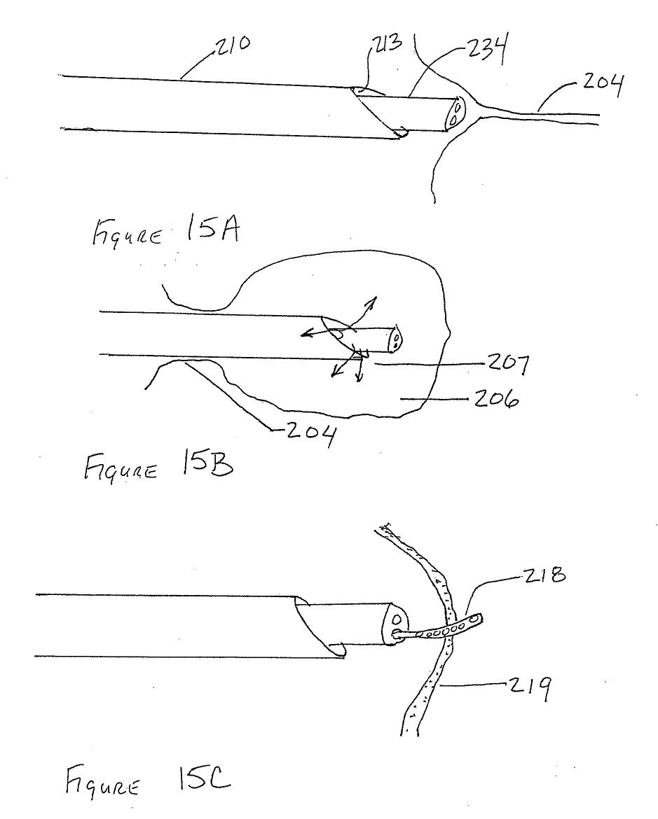

In another embodiment of the invention, the abdominal space or cavity may be accessed using a device and method that combines many aspects of other embodiments and may simplify access to the abdominal cavity. A hollow introducer sheath 200 having distal and proximal end portions is shown in FIG. 14A. The introducer sheath 200 may be rigid or flexible and may be made from polymeric materials, metallic or metal alloys such as Nitinol. The introducer sheath has a main lumen 202 that is sized for introduction of an endoscope, elongated member or obturator. The introducer sheath 200 may include a sealing member 205 that is positioned in the main lumen 202 to provide a fluid tight seal between the outside diameter of an endoscope, elongated member or obturator and the inside diameter of the main lumen. This sealing member may be a septum, membrane, o-ring, or other mechanical means to form a seal as is well known in the art. Alternatively the sealing member 205 may be a reduction in the inside diameter of the main lumen 202 that is sized to closely fit with the outside diameter of an endoscope, elongated member or obturator. The introducer sheath 200 may include an inflatable balloon 203 attached at the distal end portion that can be inflated with gas or liquid from the proximal end portion. The balloon may be sized to inflate once inside the bladder 206 to at least partially fill the bladder and anchor the introducer sheath inside the bladder during use. This may be important to provide a stable platform from which to advance the endoscope or instruments into the abdominal cavity.

The introducer sheath 200 may have an obturator placed inside the main lumen for introduction through the urethra 204 and into the bladder 206. In practice, the introducer sheath is introduced to the urethral opening and passed along the urethra 204 and into the bladder 206 similar to the placement of a Foley catheter. Once in the bladder 206, the balloon 203 can be inflated to anchor the sheath 200 inside. The introducer sheath 200 may also be used to disinfect or sterilize the bladder 206 as shown in FIG. 14B. Generally the bladder 206 is considered a sterile environment however when foreign objects such as catheters, introducers and endoscopes are introduced into the bladder 266, there exists the potential for contamination and infection. This contamination can be controlled and treated if confined to the bladder, however if this infection is introduced into the abdominal space when instruments traverse the bladder wall, the result of these infections may be more serious. Therefore once the sheath is in place a sterilizing or a disinfecting fluid 207 may be introduced through the sheath and into the bladder using the main lumen 202 or other lumens. This fluid flush is designed to at least partially disinfect or sterilize the bladder walls and the introducer sheath 200. If the end of the sheath 200 is adjacent to the intended site of bladder wall entry, particularly this portion of the bladder wall may be disinfected.

Once the sheath is in place, a hollow elongated member 210 having a proximal end portion 212 and a distal end portion 214, with a center lumen 213 that is sized for introduction of an endoscope or obturator, may be placed inside the main lumen 202 of the sheath as shown in FIGS. 14C and D. The elongated member may also have a sealing member 215 that is similar to the sealing member 205 previously described for the sheath. In this location the sealing member 215 is also designed to provide a fluid tight seal between the outside diameter of a scope or obturator and the inside diameter of elongated member center lumen 213.

This elongated member 210 may have other lumens as well that are formed integrally in the wall of the member or are formed or attached to the outside diameter of the elongated member body. In one embodiment, a utility lumen 216 is formed along the outside wall of the elongated member 210. This lumen is continuous from the proximal end portion 212 of the elongated member to a point proximal of the distal end portion 214. The end of this lumen terminates at this location so that when the elongated member is placed inside the abdominal cavity, the utility lumen 216 remains inside the bladder space. When accessing the abdominal space through the bladder wall, in order to minimize trauma and potential for leakage out of the abdominal space once any device is withdrawn from the bladder, a small initial access site is desirable. When a small access site is created, it is believed that the tissue that makes up the bladder wall 219 will collapse to its near original condition when the elongated member 210 is removed and seal the access hole without requiring secondary interventions such as stapling or suturing. Preferably the outside diameter of the elongated member should be less than 9 mm and more preferably the outside diameter should be 7 mm or less. Therefore because the length of the utility lumen 216 in this embodiment is designed to remain inside the bladder, the access site size can be minimized. Secondarily, with the utility lumen 216 positioned on the outside portion of the elongated member along a longitudinal axis offset from the longitudinal axis of the elongated member, a piercing type instrument directed through the bladder wall using this lumen may create an access site separate from the elongated member 210 access site. This may effectively reduce the overall diameter of the access site by separating one larger access site into two smaller sites each having their own small access site size.

When the elongated member 210 is positioned inside the sheath 200 as shown in FIG. 14E, the sealing member 205 may seal around the elongated member and if a scope or obturator is disposed inside the lumen of the elongated member, the sealing member 215 may seal around them.

A piercing member 218 may be positioned inside the utility lumen 216. The piercing member 218 may be a Veress needle or a trocar, wire, hypotube or other device having sufficient column strength to pierce the bladder wall when pushed distally by the operator. The piercing member 218 is designed to make an initial small diameter access to the abdominal cavity and then initiate insufflation of the cavity. In one embodiment, at least the distal portion of the piercing member 218 is hollow including a hollow tip 220 and is perforated with holes 222 that transverse the wall of the piercing member 218. These holes are designed to accommodate fluid flow between one section of the piercing member with another section of the piercing member. As shown in FIG. 14F, the piercing member 218 is positioned across the bladder wall 219 with one section of the piercing member proximal and one section positioned distal of the bladder wall 219. As shown by the arrows, fluid (gas or liquid) can pass through the bladder wall by entering a proximal hole on the interior of the bladder and exiting a distal hole located in the interior of the bladder. This process can be enhanced by forming a small chamber around the inlet side of the device thus concentrating gas build up in a smaller space. This could be achieved by advancing the cannula against the wall of the bladder.

With the access device configured as shown in FIG. 14E, with the introducer sheath positioned inside the bladder and in a preferred embodiment, an endoscope disposed inside the hollow elongated member, the bladder may be at least partially insufflated with gas introduced through the introducer sheath or the elongated member. This insufflation might provide additional tension to the bladder wall and facilitate advancement of devices through the bladder wall 219. Additionally as the bladder wall 219 is punctured with the piercing member 218 having perforated holes 222, insufflation gas will flow between the inflated bladder and into the abdominal space. This gas may be helpful to partially inflate the abdominal cavity which may provide more space for the subsequent placement of the elongated member and other instruments.

It may be important that the penetration of the bladder wall be directly visualized using the endoscope. The visualization of the target access site may help guide the proper placement position, angle, depth, and in the female avoidance of the uterus that pouches against the bladder wall, etc. of the piercing member 218 and elongated member 210. The endoscope may be extended and manipulated as necessary to insure that an adequate view of the access site is obtained. Additionally clear flushing fluid may be infused through a working channel of the endoscope or other lumens of the elongated member to clear the visual field for improved viewing.

Once the piercing member 218 has penetrated into the abdominal space, the elongated member 210 may be advanced through the bladder wall and into the abdominal cavity using the endoscope to visualize and guide the procedure. The piercing member 218 may then be withdrawn from the bladder and the utility lumen. With the elongated member in the abdominal space, the abdominal space may be further insufflated by infusing a gas through the center lumen of the elongated member, using other lumens of the elongated member or through the working channel of the endoscope. Additional instruments may be introduced to the abdominal space as needed through the endoscope or through the elongated member. Upon completion of the procedure, the elongated member 210 can be withdrawn from the bladder wall 219 and the access site will close. The introducer sheath and the elongated member are then withdrawn from the urethra.

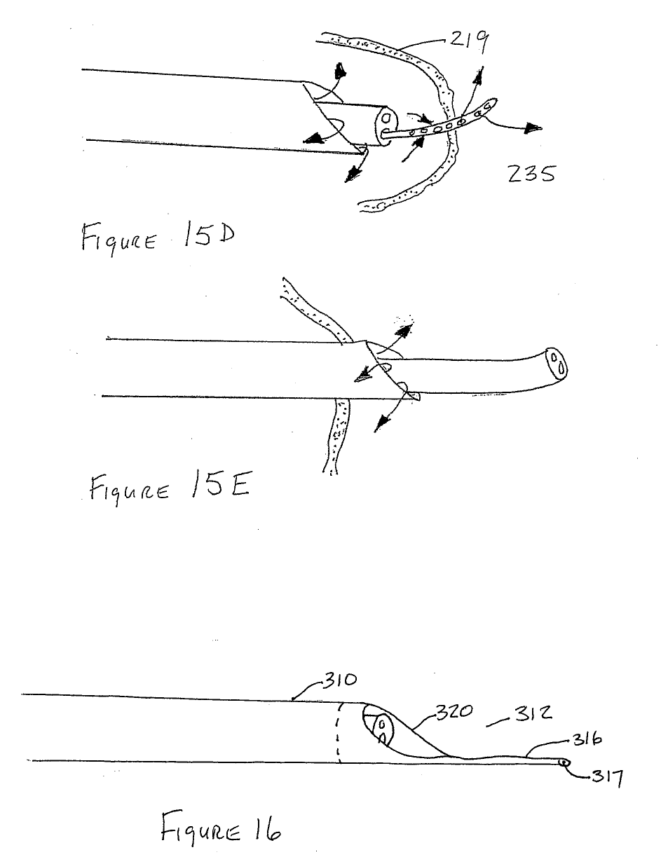

Alternatively several of the steps and device configurations previously described may be combined. In a preferred embodiment of the present invention shown in FIG. 15A, an endoscope 234 may be placed inside the central lumen 213 of the elongated member 210 and the elongated member introduced to the urethral opening and passed along the urethra 204 and into the bladder 206 using endoscopic guidance but without the use of an introducer sheath. Inthis configuration, the endoscope may provide visualization and also function as an obturator to facilitate the negotiation of the urethral pathway. Alternatively, an obturator may first be inserted into the elongated member 210 to aid insertion into the bladder 206 and then be exchanged for the endoscope. Once the tip of the elongated member has been positioned in the bladder, the bladder may be flushed with a sterilizing or a disinfecting fluid 207 to at least partially disinfect the bladder wall and the elongated member as shown in FIG. 15B. The bladder 206 may be later insufflated with gas using the center lumen 213 of the elongated member or the working channel of the endoscope.

As shown in FIG. 15C, the piercing member 218 is introduced to the access site through the working lumen of the endoscope. The piercing member 218 penetrates the bladder wall 219 releasing some of the bladder insufflation gas into the abdominal space as shown by the arrows in FIG. 15D. As shown the gas is shown entering the bladder from lumen 213 of the elongated member 210 and then entering one of the holes in the body of the hollow piercing member and exiting through a distal hole into the abdominal space 235. With the piercing member 218 across the wall 219, the elongated member 210 is inserted into the abdominal cavity using the piercing member as a guide and then the piercing member can be removed from the working lumen for the insertion of other instruments as shown in FIG. 15E. Insufflation of the abdominal cavity may be accomplished by infusing an insufflation gas through the center lumen 213 as shown by the arrows. Upon completion of the procedure, the elongated member 210 can be withdrawn from the abdominal cavity through the bladder wall 219 and the wall access site will self-close. The elongated member may then be withdrawn from the urethra.

Another embodiment of the current invention is an elongated member 310 similar to elongated member 210 but having a distal end portion 312 adapted for cavity penetration and insufflation. The distal end portion 312 has a specialized tip having a protruded nose 316. The nose is the distal termination of an inflation lumen 317 that is continuous from the proximal end to the distal end of the elongated member. The inflation lumen 317 can be used to insufflate the bladder or abdominal cavity with gas. The nose is also configured to pierce the bladder wall instead of a piercing member as previously described. The nose 316 may be inserted through the bladder wall like a needle because of its small needle like shape. As the nose 316 enters the peritoneum the nose and the tapered sidewalls 320 also facilitate the entry of the elongated member 310 into the peritoneum. The specialized tip may be integrally formed with the elongated member or could be a separate tip that is coupled to the elongated member.

This invention has been described and specific examples of the invention have been portrayed. The use of those specifics is not intended to limit the invention in anyway. Additionally, to the extent that there are variations of the invention, which are within the spirit of the disclosure or equivalent to the inventions found in the claims, it is our intent that this patent will cover those variations as well.

1. A device for trans-urinary bladder access of an anatomical cavity comprising:
an elongated member having distal and proximal end portions with a lumen extending longitudinally therein, said lumen adapted to receive an endoscope or obturator, said distal end portion of the elongated member configured to penetrate a wall of a bladder, a hollow piercing member having a sidewall, a distal end region of said member having multiple openings in said sidewall and the distal end of the member configured to penetrate the wall of the bladder.

2. The piercing member of claim 1 wherein the member is disposed in a working channel of the endoscope or in a utility lumen of the elongated member.

3. The piercing member of claim 2 further configured such that when the distal end region of said member traverses the bladder wall with at least one opening exposed to an open area on each side of the bladder, the member is adapted to facilitate fluid transfer between said open areas.

4. The utility lumen of claim 2 further comprising at least one lumen coupled to or integral with the elongated member having an open distal end, the open distal end terminating proximal to the distal end portion of the elongated member such that when a portion of the elongated member is positioned in the anatomical cavity, the distal end of the lumen is inside the bladder.

5. The device of claim 1 comprising a sealing member adapted to provide a fluid tight seal between the outside diameter of the endoscope or obturator and the inner diameter of the lumen.

6. The device of claim 1 further comprising a first expandable member positioned at the distal end portion, said first expandable member adapted to expand from a non-expanded condition to an expanded condition having a cross sectional area substantially larger than said elongated member,
the distance between the first expandable member in the expanded condition and the distal end portion is adapted to control the depth of penetration of the elongated member through the wall of the bladder.

7. The device of claim 6, whereby the expansion member is an elastic balloon.

8. The device of claim 1 further comprising:
a removable outer sheath disposed over the length of the elongated member, the sheath having a closed distal end that is adapted to be penetrated by a distal movement of the elongated member relative to the sheath.

9. The sheath of claim 8 wherein the closed distal end is clear and at least partially perforated and thereby adapted to open up when the elongated member is moved distally relative to the sheath.

10. A device for trans-urinary bladder access of an abdominal cavity comprising:
an elongated member having distal and proximal end portions with a lumen extending longitudinally therein,
a first expandable member positioned at the distal end portion, said first expandable member adapted to expand radially from a non-expanded condition to an expanded condition having a cross sectional area larger than said elongated member, the distal end portion adapted to penetrate a wall of a bladder,
and the depth of the penetration of the distal end portion of the elongated member into the cavity is controlled by the distance between a distal edge of the first expandable member in the expanded condition and the distal end portion of the elongated member.

11. The device of claim 10 further comprising a hollow obturator, having distal and proximal ends, slidably disposed within the lumen and having a penetrating member disposed at the distal end.

12. The penetrating member of claim 11 comprising an atraumatic tip configured to separate anatomical tissue or muscle layers so as to penetrate the anatomical cavity wall by spreading the tissue or muscle layers without cutting said muscle or tissue.

13. The device of claim 10 further comprising:
a second expandable member adapted to expand from a non-expanded condition to an expanded condition having a cross sectional area substantially larger than said elongated member and adapted to expand inside the cavity, said second expandable member positioned distal of the first expandable member and adapted to seal around the anatomical wall and the elongated member.

14. The device of claim 10 further comprising an inner sheath slidably disposed within the lumen and having distal and proximal ends, having an expandable member coupled to the distal end portion and adapted to expand from a non-expanded condition to an expanded condition having a cross sectional area substantially larger than said lumen,
said sheath advanceable into the anatomical cavity and the expanded expandable member adapted to seal around the anatomical wall and the elongated member to inhibit fluid leakage and anchor the elongated member at the penetration site.

15. A method of performing trans-urinary bladder access of an abdominal cavity comprising:
positioning a tip of a hollow elongated member through a urethra and against a wall of a bladder, the elongated member adapted to receive an endoscope,
disinfecting at least a portion of the bladder by infusing a disinfecting fluid through the hollow portion of the elongated member,
and puncturing the wall with a hollow piercing member and advancing the elongated member through the wall and into the cavity.

16. The method of claim 15 wherein puncturing the wall includes insufflating the bladder with gas, releasing the gas from the bladder through the hollow piercing member and into the abdominal cavity so that the abdominal cavity is at least partially insufflated.

17. The method of claim 16 wherein the piercing member is delivered to the bladder wall through a working lumen of the endoscope or through a utility lumen of the elongated member.

18. The method of claim 15 further comprising extending an endoscope disposed in the hollow portion of the elongated member and directly visualizing the steps of puncturing the wall and advancing the elongated member.

19. The method of claim 18 wherein the abdominal cavity is further insufflated by infusing a gas through the hollow portion of the elongated member.

20. The method of claim 15 wherein advancing the elongated member through the wall further comprises extending an obturator, said obturator having a blunt tip that is adapted to spread tissue without cutting and disposed in the elongated member, through the bladder wall tissue without cutting the tissue.

21. The method of claim 20 wherein advancing the elongated member through the wall further comprises sequentially advancing the obturator until the blunt tip separates at least one tissue plane aligned in one axis, rotating the blunt tip until the blunt tip separates at least one tissue plane aligned in a different axis, and repeating these steps until the obturator is in the abdominal cavity. A method of accessing an abdominal space through a bladder wall comprising:
positioning an introducer sheath into the bladder, said introducer sheath having an inflatable anchor balloon,
inflating said anchor balloon to maintain the position of the sheath inside the bladder, flushing a disinfecting fluid through the sheath to at least partially disinfect the bladder wall,
inserting a cannula, with an endoscope moveably disposed within, into the sheath and advancing said cannula to the wall of the bladder,
advancing the cannula through the bladder wall and into the abdominal space,
and inflating the abdominal space with gas.

22. The method of claim 21 further comprising prior to advancing said cannula through the bladder wall, inserting a piercing member into a lumen of the cannula or into a working channel of the endoscope and through the bladder wall and introducing a gas through the piercing member to at least partially inflate the abdominal space with a gas,

23. The method of claim 22 wherein the piercing member is a veress-type needle.

24. A method of sterilizing an anatomical cavity access site having a wall, said wall having an outer region comprising:
positioning a distal end of a hollow elongated member against a portion of the outer region, the elongated member having a piston-like obturator moveably disposed within such that a distal end of the obturator lies proximal of the distal end of a hollow elongated member thus forming an open space, said obturator having a sealing member adapted to provide a fluid seal between the obturator and the inner diameter of the elongated member,
infusing a fluid for sterilizing tissue into said open space and advancing the piston-like obturator toward the distal end of the elongated member to reduce the open space and force the sterilizing fluid through the distal end of the elongated member and into and around a portion of the outer region of the wall.

25. The method of claim 24 further comprising advancing the elongated member through the anatomical cavity wall.
